(19) United States
(12) Patent Application Publication
(10) Pub. No.: US 2026/0315261 A1
(43) Pub. Date: Oct. 15, 2026

(54) ABDOMINAL BINDER

(71) Applicant: Shenzhen Lute Jiacheng Supply Chain Management Co., Ltd., Shenzhen (CN)

(72) Inventors: Zijuan Qi, Shenzhen (CN); Ping Yan, Shenzhen (CN)

(21) Appl. No.: 19/190,827

(22) Filed: Apr. 28, 2025

(30) Foreign Application Priority Data
Apr. 15, 2025 (CN) 202520715531.2

(51) Int. Cl. A61F 5/03

(52) U.S. Cl. CPC A61F 5/03

(57) ABSTRACT

The present application discloses an abdominal binder, comprising: a waist support portion configured to be positioned at a user's waist region and to extend toward both sides of the user when worn; a first abdominal compression portion connected to a first side of the waist support portion; and a second abdominal compression portion connected to a second side of the waist support portion; wherein the first abdominal compression portion and the second abdominal compression portion are detachably connected and provide a compressive force to the user's abdomen when connected; wherein the abdominal binder is at least partially made of a sheet material comprising a first outer layer and a second outer layer, and an adhesive layer disposed between the first outer layer and the second outer layer.

### CROSS-REFERENCE TO RELATED APPLICATIONS

The present application claims priority to Chinese Patent Application No. 202520715531.2, filed on Apr. 15, 2025, the entire contents of which are incorporated herein by reference.

### TECHNICAL FIELD

The present application relates to the technical field of maternal and baby products, particularly to an abdominal binder.

### BACKGROUND

With increasing public attention to health and body shape, various types of abdominal binders have emerged in the market and are widely used in fields such as weight loss, postpartum recovery, and athletic support. Traditional abdominal binders mainly achieve abdominal compression by tightening the waist and abdominal regions to improve abdominal appearance and assist in abdominal muscle recovery. Common abdominal binders generally include a waist support portion and an abdominal compression portion, which are typically integrated by sewing or bonding techniques. However, there remain several issues to be addressed in the existing technologies, particularly in terms of stitching techniques, elasticity performance, and compression effectiveness.

In the current manufacturing processes, abdominal binders are typically assembled using sewing methods, which can result in uneven seams that adversely affect wearing comfort and aesthetic appearance. Furthermore, the stitched portions may cause discomfort during use, making it difficult for the wearer to wear the binder for extended periods. In addition, the existing abdominal binders often lack sufficient elasticity performance, making it difficult to provide uniform and effective compressive force, thereby resulting in suboptimal abdominal compression effects. Particularly in the abdominal region, the binder frequently fails to apply consistent pressure, which compromises the intended shaping or recovery outcomes.

Accordingly, in view of the issues in the prior art such as uneven stitching, insufficient elasticity, and poor compression performance, there is an urgent need for a novel abdominal binder structure to improve wearing comfort, elasticity performance, and compression effectiveness, so as to better meet user needs.

### SUMMARY

In view of the deficiencies in the prior art, an embodiment of this application provides an abdominal binder that can solve the problems of poor elasticity, ineffective abdominal compression, uneven appearance, and difficulties in cutting during production.

To address the above technical problems, one embodiment of the present application provides an abdominal binder, comprising: a waist support portion configured to be positioned at a user's waist region and to extend toward both sides of the user when worn; a first abdominal compression portion connected to a first side of the waist support portion and configured to extend along the abdomen when worn; and a second abdominal compression portion connected to a second side of the waist support portion and configured to extend along the abdomen when worn; wherein the first abdominal compression portion and the second abdominal compression portion are detachably connected and provide a compressive force to the user's abdomen when connected; wherein the abdominal binder is at least partially made of a sheet material comprising a first outer layer and a second outer layer, and an adhesive layer disposed between the first outer layer and the second outer layer, the first and second outer layers being bonded by the adhesive layer.

In one embodiment, the adhesive layer comprises a first adhesive sub-layer, a cushioning layer, and a second adhesive sub-layer sequentially stacked.

In one embodiment, the cushioning layer is a sponge layer or gauze layer.

In one embodiment, the cushioning layer is provided with ventilation holes.

In one embodiment, the first outer layer is a polyester-spandex fabric and the second outer layer is a nylon-spandex fabric.

In one embodiment, wherein the adhesive layer is a glue layer.

In one embodiment, the first adhesive sub-layer and the second adhesive sub-layer are glue layers.

In one embodiment, wherein the first abdominal compression portion and the second abdominal compression portion are detachably connected by hook-and-loop fastener, wherein one of the first and second abdominal compression portions is provided with a hook surface and the other is provided with a loop surface.

In one embodiment, wherein the hook-and-loop fastener includes a notch.

In one embodiment, wherein the hook-and-loop fastener is sewn onto the first and/or second abdominal compression portion by stitching.

In one embodiment, the stitching comprises a first stitching line located at an edge region of the hook-and-loop fastener and a second stitching line located at a central region of the hook-and-loop fastener.

In one embodiment, the first abdominal compression portion comprises at least a first compression band and a second compression band, the first and second compression bands extending laterally side-by-side along the abdomen when worn, being independently and detachably connectable to the second abdominal compression portion, and having individually adjustable tightness by changing the connection positions.

In one embodiment, the first compression band and the second compression band at least partially overlap.

In one embodiment, one of the inner surface of the first compression band and the outer surface of the second compression band is provided with a loop surface of a hook-and-loop fastener, and the other is provided with a hook surface, so that the overlapping region adheres together when worn.

In one embodiment, the abdominal binder is embedded with anti-curling support strip extending in a vertical direction when worn.

In one embodiment, the anti-curling support strip gradually decrease in thickness from the middle toward both ends.

In one embodiment, the anti-curling support strip gradually decrease in width from the middle toward both ends.

In one embodiment, the waist support portion comprises a back panel and first and second side panels extending respectively from opposite sides of the back panel, and the back panel has a greater thickness than each of the side panels.

In one embodiment, the first abdominal compression portion has a greater thickness than the first and second side panels, and the second abdominal compression portion has a greater thickness than the first and second side panels.

In one embodiment, the first abdominal compression portion has greater elasticity than the second abdominal compression portion.

In one embodiment, the second abdominal compression portion is provided with at least one lifting handle.

In one embodiment, the second abdominal compression portion is provided with two lifting handles, the two lifting handles being positioned at different height levels of the second abdominal compression portion.

In one embodiment, the second abdominal compression portion substantially covers the user's entire front abdominal region.

In one embodiment, the hook-and-loop fastener on the second abdominal compression portion substantially covers the user's entire front abdominal region.

In one embodiment, the first abdominal compression portion is provided with hook surfaces of a hook-and-loop fastener, wherein the height of the hooks is smaller than the thickness of the first abdominal compression portion.

In one embodiment, an edge spacing is maintained between the periphery of the second abdominal compression portion and the edge of the hook-and-loop fastener disposed thereon.

In one embodiment, the hook-and-loop fastener on the second abdominal compression portion is secured via stitching comprising first stitching lines located at edge regions of the hook-and-loop fastener and second stitching lines located at a central region of the hook-and-loop fastener, the second stitching lines having a wave-shaped pattern.

To address the above technical problems, one embodiment of the present application provides an abdominal binder, comprising: a waist support portion configured to be positioned at a user's waist region and to extend toward both sides of the user when worn; a first abdominal compression portion connected to a first side of the waist support portion and configured to extend along the abdomen when worn; and a second abdominal compression portion connected to a second side of the waist support portion and configured to extend along the abdomen when worn; wherein the first and second abdominal compression portions are detachably connected to provide compressive force to the user's abdomen when connected; wherein the first and second abdominal compression portions are detachably connected by hook-and-loop fastener, with one provided with a hook surface and the other with a loop surface, and the hook-and-loop fastener includes a notch.

To address the above technical problem, one embodiment of the present application provides an abdominal binder comprising: a waist support portion configured to be positioned at a user's waist region and extend toward both sides of the user when worn; a first abdominal compression portion connected to a first side of the waist support portion and configured to extend along the abdomen when worn; and a second abdominal compression portion connected to a second side of the waist support portion and configured to extend along the abdomen when worn; wherein the first abdominal compression portion and the second abdominal compression portion are detachably connected to provide compressive force to the user's abdomen when connected; wherein the first abdominal compression portion and the second abdominal compression portion are detachably connected via a hook-and-loop fastener, with one of the first abdominal compression portion and the second abdominal compression portion being provided with a hook surface of the hook-and-loop fastener and the other being provided with a loop surface; and wherein the hook-and-loop fastener is sewn onto the first abdominal compression portion and/or the second abdominal compression portion via a stitching comprising a first stitching line located at an edge region of the hook-and-loop fastener and a second stitching line located at a central region of the hook-and-loop fastener.

To address the above technical problems, one embodiment of the present application provides an abdominal binder, comprising: a waist support portion configured to be positioned at a user's waist region and to extend toward both sides of the user when worn; a first abdominal compression portion connected to a first side of the waist support portion and configured to extend along the abdomen when worn; and a second abdominal compression portion connected to a second side of the waist support portion and configured to extend along the abdomen when worn; wherein the first and second abdominal compression portions are detachably connected to provide a compressive force to the user's abdomen when connected; wherein the first abdominal compression portion comprises at least a first compression band and a second compression band, the first and second compression bands extending laterally side-by-side along the abdomen when worn, being independently and detachably connectable to the second abdominal compression portion, and having individually adjustable tightness by changing the connection positions.

In one embodiment, the first and second compression bands at least partially overlap.

In one embodiment, one of the inner surface of the first compression band and the outer surface of the second compression band is provided with the loop surface of a hook-and-loop fastener, and the other is provided with the hook surface, so that the overlapping region adheres together when worn.

To address the above technical problems, one embodiment of the present application provides an abdominal binder comprising: a waist support portion configured to be positioned at a user's waist region and extend toward both sides of the user when worn; a first abdominal compression portion connected to a first side of the waist support portion and configured to extend along the abdomen when worn; and a second abdominal compression portion connected to a second side of the waist support portion and configured to extend along the abdomen when worn; wherein the first abdominal compression portion and the second abdominal compression portion are detachably connected to provide compressive force to the user's abdomen when connected; and wherein the abdominal binder is embedded with an anti-curling support strip extending vertically when the abdominal binder is worn.

In one embodiment, the anti-curling support strip gradually decreases in thickness from its middle portion toward both ends.

In one embodiment, the anti-curling support strip gradually decreases in width from its middle portion toward both ends.

To address the above technical problems, one embodiment of the present application provides an abdominal binder comprising: a waist support portion configured to be positioned at a user's waist region and extend toward both sides of the user when worn; a first abdominal compression portion connected to a first side of the waist support portion and configured to extend along the abdomen when worn; and a second abdominal compression portion connected to a second side of the waist support portion and configured to extend along the abdomen when worn; wherein the first abdominal compression portion and the second abdominal compression portion are detachably connected to provide compressive force to the user's abdomen when connected; and wherein the waist support portion comprises a back panel and first and second side panels extending from opposite sides of the back panel respectively, the back panel having a greater thickness than both the first side panel and the second side panel.

In one embodiment, the first abdominal compression portion has a greater thickness than both the first side panel and the second side panel, and the second abdominal compression portion has a greater thickness than both the first side panel and the second side panel.

To address the above technical problems, one embodiment of the present application provides an abdominal binder comprising: a waist support portion configured to be positioned at a user's waist region and extend toward both sides of the user when worn; a first abdominal compression portion connected to a first side of the waist support portion and configured to extend along the abdomen when worn; and a second abdominal compression portion connected to a second side of the waist support portion and configured to extend along the abdomen when worn; wherein the first abdominal compression portion and the second abdominal compression portion are detachably connected to provide compressive force to the user's abdomen when connected; and wherein the first abdominal compression portion has greater elasticity than the second abdominal compression portion.

According to an embodiment of the present application, the abdominal binder includes: a waist support portion configured to be positioned at the user's waist region when worn and to extend toward both sides of the user; a first abdominal compression portion connected to a first side of the waist support portion and configured to extend along the abdomen when worn; and a second abdominal compression portion connected to a second side of the waist support portion and configured to extend along the abdomen when worn. The first abdominal compression portion and the second abdominal compression portion are detachably connected, and when connected, provide a compressive force to the user's abdomen. The abdominal binder is at least partially made of a sheet material, the sheet material including a first outer layer, a second outer layer, and an adhesive layer disposed between the first outer layer and the second outer layer, wherein the first outer layer and the second outer layer are bonded together via the adhesive layer. This configuration improves the elasticity and surface smoothness of the abdominal binder, thereby enhancing the abdominal compression effect and wearing comfort, while also facilitating sheet cutting and simplifying the manufacturing process.

### BRIEF DESCRIPTION OF THE DRAWINGS

To more clearly illustrate the technical solutions in the embodiments of the present application or the prior art, the following provides a brief description of the accompanying drawings required for describing the embodiments or the prior art. It is evident that the drawings in the following description are merely some embodiments of the present application, and other drawings can be obtained by those of ordinary skill in the art based on the structures shown in these drawings without creative effort.

FIG. 1 is a schematic view showing the outer surface of an abdominal binder in an unfolded state according to one embodiment of the present application;

FIG. 2 is a schematic view showing the inner surface of the abdominal binder in an unfolded state according to one embodiment of the present application;

FIG. 3 is a schematic view showing the abdominal binder in a worn state according to one embodiment of the present application;

FIG. 4 is a schematic view showing the outer surface of an abdominal binder in an unfolded state according to another embodiment of the present application;

FIG. 5 is a schematic view illustrating the laminated structure of a sheet material according to one embodiment of the present application;

FIG. 6 is a schematic view illustrating the laminated structure of a sheet material according to another embodiment of the present application.

The implementation, functional features, and advantages of the present application will be further described in conjunction with the embodiments and with reference to the accompanying drawings.

### DETAILED DESCRIPTION OF THE EMBODIMENTS

In the present application, the terms “arranged,” “provided with,” and “connected” should be understood broadly. For example, a connection may be fixed, detachable, or integrally constructed; it may be mechanical or electrical; it may be direct or indirectly connected through an intermediary, or it may refer to internal communication between two devices, components, or constituent parts. Those of ordinary skill in the art can understand the specific meanings of these terms in the context of the present application based on specific circumstances.

The terms “center,” “longitudinal,” “transverse,” “length,” “width,” “thickness,” “upper,” “lower,” “front,” “rear,” “left,” “right,” “vertical,” “horizontal,” “top,” “bottom,” “inner,” “outer,” “radial,” and “circumferential” are used to describe orientations or positional relationships based on the orientations or positional relationships shown in the accompanying drawings. These terms are used solely for the purpose of facilitating the description of the present application and simplifying the description, and do not indicate or imply that the referenced devices or components must have a specific orientation or be constructed and operated in a specific orientation. Therefore, these terms should not be construed as limiting the present application.

Furthermore, the terms “first” and “second” are used for descriptive purposes only and should not be construed as indicating or implying relative importance or implicitly specifying the quantity of the indicated technical features. Thus, features defined by “first” or “second” may explicitly or implicitly include at least one such feature. In the description of the present application, the term “plurality” means at least two, such as two, three, etc., unless explicitly stated otherwise.

Additionally, some of the above terms may be used to indicate meanings other than orientation or positional relationships. For example, the term “upper” may, in some cases, also be used to indicate a certain dependency or connection relationship. Those of ordinary skill in the art can understand the specific meanings of these terms in the context of the present application based on specific circumstances.

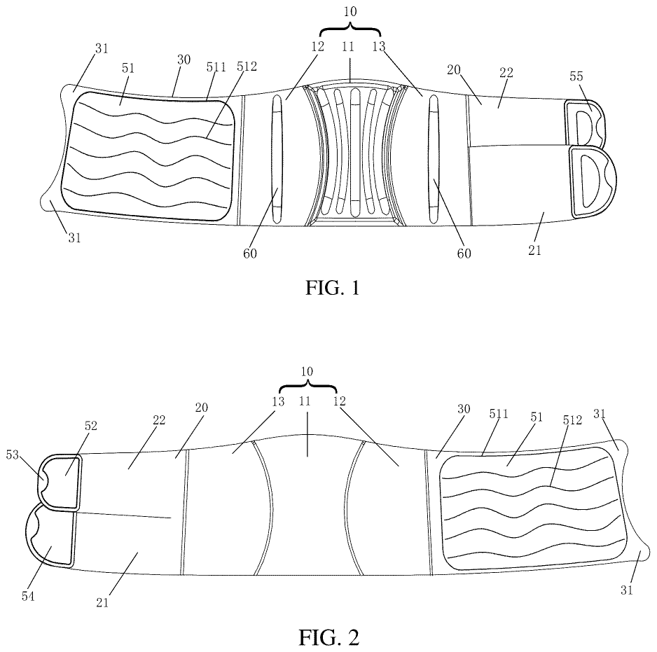

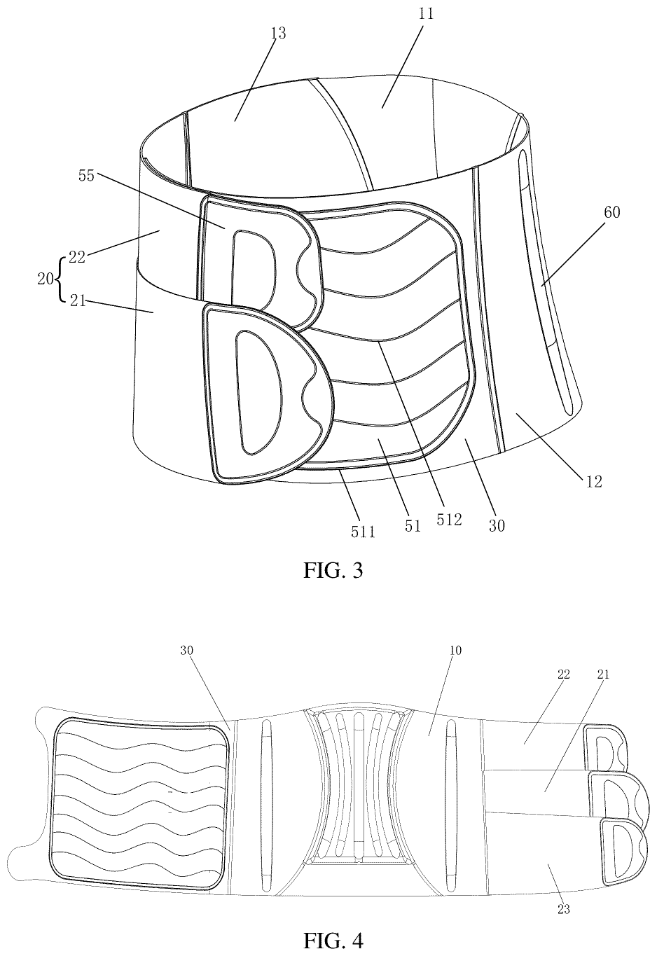

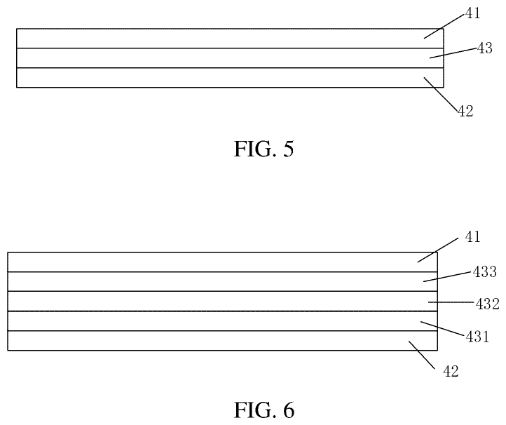

Referring to FIGS. 1 to 6, FIG. 1 is a schematic view showing the outer surface of an abdominal binder in an unfolded state according to one embodiment of the present application; FIG. 2 is a schematic view showing the inner surface of the abdominal binder in an unfolded state according to one embodiment of the present application; FIG. 3 is a schematic view showing the abdominal binder in a worn state according to one embodiment of the present application; FIG. 4 is a schematic view showing the outer surface of an abdominal binder in an unfolded state according to another embodiment of the present application; FIG. 5 is a schematic view illustrating the laminated structure of a sheet material according to one embodiment of the present application; and FIG. 6 is a schematic view illustrating the laminated structure of a sheet material according to another embodiment of the present application.

As shown in FIGS. 1, 2, and 3, in one embodiment of the present application, the abdominal binder comprises: a waist support portion 10 configured to be positioned at a user's waist region and to extend toward both sides of the user when worn; a first abdominal compression portion 20 connected to a first side of the waist support portion 10 and configured to extend along the abdomen when worn; and a second abdominal compression portion 30 connected to a second side of the waist support portion 10 and configured to extend along the abdomen when worn; wherein the first abdominal compression portion 20 and the second abdominal compression portion 30 are detachably connected and provide a compressive force to the user's abdomen when connected; wherein the first abdominal compression portion 20 and the second abdominal compression portion 30 are configured to allow adjustment of tightness by changing the connection positions of the detachable connection; and wherein the detachable connection means include, but are not limited to: hook-and-loop fastener, hook-and-eye fastening structures, and snap fastening structures.

As shown in FIGS. 5 and 6, the abdominal binder is at least partially made of a sheet material comprising a first outer layer 41 and a second outer layer 42, and an adhesive layer 43 disposed between the first outer layer 41 and the second outer layer 42, the first outer layer 41 and the second outer layer 42 being bonded by the adhesive layer 43. Through this configuration, the adhesive layer between the first outer layer 41 and the second outer layer 42 can enhance the overall elasticity of the abdominal binder, thereby improving the abdominal compression effect with greater tightening force. Moreover, the abdominal binder becomes more uniform and can be freely cut into desired shapes, which facilitates edge contour design of the abdominal binder.

In one embodiment, the adhesive layer 43 comprises a first adhesive sub-layer 431, a cushioning layer 432, and a second adhesive sub-layer 433 sequentially stacked. In this configuration, the cushioning layer 432 embedded within the adhesive layer increases both the overall thickness and softness of the abdominal binder, thereby providing enhanced comfort during wear. In an alternative embodiment, the adhesive layer may consist solely of a glue layer without any embedded cushioning layer. The embodiments of the present application are not limited in this regard.

The material of the glue layer may include, but is not limited to, hot-melt adhesive, polyurethane adhesive, solvent-based adhesive, silicone adhesive, acrylic adhesive, or two-component adhesive. The embodiments of the present application are not limited in this regard.

In one embodiment, the cushioning layer 432 is a sponge layer or gauze layer. The cushioning layer 432 is configured to increase both thickness and softness of the abdominal binder, thereby improving wearing comfort.

In one embodiment, the cushioning layer 432 is formed with ventilation holes. For example, the cushioning layer 432 may be a mesh fabric layer. While increasing thickness, the cushioning layer 432 ensures breathability through the ventilation holes.

In one embodiment, the first outer layer 41 is a polyester-spandex fabric, serving as the outermost layer when the abdominal binder is worn, and the second outer layer 42 is a nylon-spandex fabric, serving as the innermost layer during wear.

In one embodiment, the first adhesive sub-layer 431 and the second adhesive sub-layer 433 are glue layers. The polyester-spandex fabric is a blended or interwoven textile comprising polyester and spandex, characterized by waterproofing, wear resistance, high elasticity, and wrinkle resistance. The nylon-spandex fabric is an elastic blended material combining nylon and spandex, which maintains nylon's tensile strength and spandex's superior elasticity while providing moisture-wicking properties and sweat corrosion resistance.

As shown in FIGS. 1, 2, and 3, in one embodiment, the first abdominal compression portion 20 and the second abdominal compression portion 30 are detachably connected via a hook-and-loop fastener, wherein one of the first abdominal compression portion 20 and the second abdominal compression portion 30 is provided with a hook surface of the hook-and-loop fastener, and the other is provided with a loop surface. Optionally, the first abdominal compression portion 20 is connected to the right side of the waist support portion 10, while the second abdominal compression portion 30 is connected to the left side of the waist support portion 10. The second abdominal compression portion 30 is provided with a loop surface 51, and the first abdominal compression portion 20 is provided with a hook surface 52.

This hook-and-loop configuration enables convenient connection and detachment between the first and second abdominal compression portions 20, 30, allowing the user to freely adjust abdominal compression as needed. The design enhances both flexibility and comfort of the abdominal binder. The hook-and-loop fastener may be disposed at an end portion or adjacent to an end portion of the first and second abdominal compression portions 20, 30.

In one embodiment, the hook-and-loop fastener 52 is formed with a notch 53. Optionally, the hook surface 52 on the first abdominal compression portion 20 includes the notch 53. The notch 53 prevents friction between the user's fingers and the hook-and-loop fastener during wearing, thereby improving operational comfort.

In one embodiment, the hook-and-loop fastener is sewn onto the first abdominal compression portion 20 and/or the second abdominal compression portion 30 by stitching. The stitching comprises a first stitching line 511 located at an edge region of the hook-and-loop fastener 51 and a second stitching line 512 located at a central region of the hook-and-loop fastener 51. This stitching configuration prevents adhesive failure of the hook-and-loop fastener caused by repeated washing, while the second stitching line 512 at the central region prevents delamination of the hook-and-loop fastener due to prolonged peeling forces.

As shown in FIGS. 1, 2 and 3, in one embodiment, the first abdominal compression portion 20 comprises at least a first compression band 21 and a second compression band 22. The first compression band 21 and the second compression band 22 extend laterally side-by-side along the abdomen when worn, being independently and detachably connectable to the second abdominal compression portion 30, and having individually adjustable tightness by changing the connection positions. The tightness of each of the first compression band 21 and the second compression band 22 can be individually adjusted by changing their connection positions with the second abdominal compression portion 30. This configuration allows zoned pressure adjustment at the positions of the first compression band 21 and the second compression band 22, thereby improving overall wearing comfort of the abdominal binder.

In one embodiment, the first compression band 21 and the second compression band 22 at least partially overlap. This overlapping arrangement prevents the user's bulging flesh from protruding between the first compression band 21 and the second compression band 22, avoiding stomach pinching while enhancing both comfort and aesthetic appearance during wear.

As shown in FIGS. 1, 2 and 3, in one embodiment, one of the inner surface of the first compression band 21 and the outer surface of the second compression band 22 is provided with a loop surface of a hook-and-loop fastener, and the other is provided with a hook surface, so that the first compression band 21 and the second compression band 22 adhere together in their overlapping region when worn. Optionally, the inner surface of the first compression band 21 is provided with a hook surface 54, and the outer surface of the second compression band 22 is provided with a loop surface 55. This configuration ensures secure attachment between the first compression band 21 and the second compression band 22, improving surface smoothness of the abdominal binder during wear.

In one embodiment, the first compression band 21 is positioned at least partially below the second compression band 22 when worn, and the length of the first compression band 21 is greater than that of the second compression band 22. This configuration, where the lower first compression band 21 is made longer than the upper second compression band 22, enhances wearing comfort.

As shown in FIG. 4, in another embodiment, the first abdominal compression portion 20 further includes a third compression band 23. The first compression band 21, second compression band 22 and third compression band 23 extend laterally side-by-side along the abdomen when worn, and are independently and detachably connectable to the second abdominal compression portion 30. Each of the first compression band 21, second compression band 22 and third compression band 23 has individually adjustable tightness by changing their connection positions with the second abdominal compression portion 30. This three-band configuration accommodates users with longer torso lengths. It should be understood that other embodiments may include four or more compression bands, as the embodiments of the present application are not limited in this regard. Optionally, the third compression band 23 has a length smaller than that of the first compression band 21 but greater than that of the second compression band 22, thereby improving wearing comfort and ergonomic design.

As shown in FIGS. 1, 2 and 3, in one embodiment, the abdominal binder is embedded with an anti-curling support strip 60 that extends substantially vertically when the abdominal binder is worn. In one embodiment, the anti-curling support strip 60 gradually decreases in thickness from its middle portion toward both ends. In one embodiment, the anti-curling support strip 60 gradually decreases in width from its middle portion toward both ends. This design, where both thickness and width taper from the middle to the ends, effectively improves comfort and fit of the abdominal binder, preventing excessive stiffness at the ends that could compromise comfort. The anti-curling support strip 60 may be made of thermoplastic polyurethane, silicone, or similar materials.

As shown in FIGS. 1, 2 and 3, in one embodiment, the waist support portion 10 comprises a back panel 11 and first and second side panels 12, 13 extending respectively from opposite sides of the back panel 11, wherein the back panel 11 has a greater thickness than both the first side panel 12 and the second side panel 13.

In one embodiment, the first abdominal compression portion 20 has a greater thickness than both the first side panel 12 and the second side panel 13. The second abdominal compression portion 30 has a greater thickness than both the first side panel 12 and the second side panel 13.

The relatively thinner first and second side panels 12, 13 enhance lateral breathability, preventing excessive heat buildup during wear while maintaining dryness and comfort. The thicker first and second abdominal compression portions 20, 30 provide stronger support to help shape the abdominal curve, thereby improving wearing comfort. This optimized thickness distribution effectively alleviates pressure, avoiding excessive compression on the wearer's abdomen and back. The thickest back panel 11 offers superior support and comfort with its soft yet firm structure.

In one embodiment, the first abdominal compression portion 20 has greater elasticity than the second abdominal compression portion 30. During wear, the second abdominal compression portion 30 positioned as the inner layer first flattens the abdomen with its lower elasticity, while the outer first abdominal compression portion 20 then tightens with its higher elasticity.

In one embodiment, the second abdominal compression portion 30 is provided with at least one lifting handle 31 for easier donning.

In one embodiment, the second abdominal compression portion 30 is provided with two lifting handles 31, the two lifting handles 31 are provided on the second abdominal compression portion 30 at different vertical positions, corresponding respectively to connection points with the first compression band 21 and second compression band 22 for optimal force application.

In one embodiment, the second abdominal compression portion 30 substantially covers the wearer's entire front abdominal region, facilitating initial flattening.

In one embodiment, the hook-and-loop fastener 51 on the second abdominal compression portion 30 substantially covers the entire front abdominal region, allowing wider adjustment range for different postpartum stages.

In one embodiment, the hook surface 52 on the first abdominal compression portion 20 has hook elements with heights smaller than the thickness of the first abdominal compression portion 20 itself, reducing skin friction for enhanced comfort.

In one embodiment, an edge spacing is maintained between the periphery of the second abdominal compression portion 30 and the edge of the hook-and-loop fastener 51 disposed thereon. This configuration prevents excessive thickness at the abdominal binder's edges, thereby improving both fit and comfort along the peripheral regions.

In one embodiment, the hook-and-loop fastener 51 on the second abdominal compression portion 30 is secured via stitching, comprising: first stitching lines 511 located at edge regions of the hook-and-loop fastener 51 and second stitching lines 512 locate data central region of the hook-and-loop fastener 51, wherein the second stitching lines 512 having a wave-shaped pattern.

The wave-shaped configuration of the second stitching lines 512 accommodates transverse stretching of the second abdominal compression portion 30 during use, effectively preventing rupture of the stitching under tension.

The embodiments of the present application provide an abdominal binder comprising: a waist support portion configured to be positioned at a user's waist region and extend toward both sides of the user when worn; a first abdominal compression portion connected to a first side of the waist support portion and configured to extend along the abdomen when worn; and a second abdominal compression portion connected to a second side of the waist support portion and configured to extend along the abdomen when worn; wherein the first abdominal compression portion and the second abdominal compression portion are detachably connected to provide compressive force to the user's abdomen when connected; and wherein the abdominal binder is at least partially made of a sheet material comprising a first outer layer, a second outer layer, and an adhesive layer disposed between the first and second outer layers, the first and second outer layers being bonded via the adhesive layer, thereby enhancing the elasticity and surface smoothness of the abdominal binder to improve abdominal compression effect and wearing comfort, while facilitating cutting of the sheet material and simplifying manufacturing process.

To make the objectives, technical solutions, and advantages of the present application clearer, the following further describes the present application in detail with reference to the accompanying drawings and embodiments. It should be understood that the specific embodiments described herein are merely intended to explain the present application and are not intended to limit the present application.

The above descriptions are only specific implementations of the present application. It should be noted that, for those of ordinary skill in the art, various modifications and refinements can be made without departing from the principles of the present application. These modifications and refinements should also be regarded as falling within the protection scope of the present application.

What is claimed is:

1. An abdominal binder, comprising:
a waist support portion configured to be positioned at a user's waist region and to extend toward both sides of the user when worn;
a first abdominal compression portion connected to a first side of the waist support portion and configured to extend along the abdomen when worn; and
a second abdominal compression portion connected to a second side of the waist support portion and configured to extend along the abdomen when worn;
wherein the first abdominal compression portion and the second abdominal compression portion are detachably connected and provide a compressive force to the user's abdomen when connected;
the first abdominal compression portion has greater elasticity than the second abdominal compression portion, such that when worn, the second abdominal compression portion, as an inner layer, flattens the abdomen with its lower elasticity, and the first abdominal compression portion, as an outer layer, subsequently tightens with its greater elasticity.

2. (canceled)

3. (canceled)

4. (canceled)

5. The abdominal binder of claim 1, wherein the first abdominal compression portion and the second abdominal compression portion are detachably connected by hook-and-loop fastener, wherein one of the first and second abdominal compression portions is provided with a hook surface and the other is provided with a loop surface.

6. The abdominal binder of claim 5, wherein the hook-and-loop fastener includes a notch.

7. The abdominal binder of claim 6, wherein the hook-and-loop fastener is sewn onto the first and/or second abdominal compression portion by stitching.

8. The abdominal binder of claim 7, wherein the stitching comprises a first stitching line located at an edge region of the hook-and-loop fastener and a second stitching line located at a central region of the hook-and-loop fastener.

9. The abdominal binder of claim 1, wherein the first abdominal compression portion comprises at least a first compression band and a second compression band, the first and second compression bands extending laterally side-by-side along the abdomen when worn, being independently and detachably connectable to the second abdominal compression portion, the first and second compression bands each having a plurality of selectable connection positions for detachably connecting to the second abdominal compression portion, and having individually adjustable tightness by changing the connection positions.

10. The abdominal binder of claim 9, wherein the first compression band and the second compression band at least partially overlap, forming an overlapping region.

11. The abdominal binder of claim 10, wherein the first compression band has an inner surface and an outer surface, the second compression band has an inner surface and an outer surface, one of the inner surface of the first compression band and the outer surface of the second compression band is provided with a loop surface of a hook-and-loop fastener, and the other is provided with a hook surface, so that the overlapping region adheres together when worn.

12. The abdominal binder of claim 1, wherein the abdominal binder is embedded with anti-curling support strip extending in a vertical direction when worn.

13. (canceled)

14. (canceled)

15. (canceled)

16. (canceled)

17. An abdominal binder, comprising:
a waist support portion configured to be positioned at a user's waist region and to extend toward both sides of the user when worn;
a first abdominal compression portion connected to a first side of the waist support portion and configured to extend along the abdomen when worn; and
a second abdominal compression portion connected to a second side of the waist support portion and configured to extend along the abdomen when worn;
wherein the first and second abdominal compression portions are detachably connected to provide compressive force to the user's abdomen when connected;
wherein the first and second abdominal compression portions are detachably connected by hook-and-loop fastener, with one provided with a hook surface and the other with a loop surface, and the hook-and-loop fastener includes a notch, the notch having a width sufficient to accommodate at least one finger of a user; wherein the notch is provided on the hook surface of the hook-and-loop fastener on the first abdominal compression portion, the notch being configured to prevent friction between a user's fingers and the hook-and-loop fastener during donning of the abdominal binder.

18. An abdominal binder, comprising:
a waist support portion configured to be positioned at a user's waist region and to extend toward both sides of the user when worn;
a first abdominal compression portion connected to a first side of the waist support portion and configured to extend along the abdomen when worn; and
a second abdominal compression portion connected to a second side of the waist support portion and configured to extend along the abdomen when worn;
wherein the first and second abdominal compression portions are detachably connected to provide a compressive force to the user's abdomen when connected;
wherein the first abdominal compression portion comprises at least a first compression band and a second compression band, the first and second compression bands extending laterally side-by-side along the abdomen when worn, being independently and detachably connectable to the second abdominal compression portion, the first and second compression bands each having a plurality of selectable connection positions for detachably connecting to the second abdominal compression portion, and having individually adjustable tightness by changing the connection positions;
wherein, when worn, the first compression band is positioned at least partially below the second compression band, and the first compression band has a length greater than a length of the second compression band.

19. The abdominal binder of claim 18, wherein the first and second compression bands at least partially overlap, forming an overlapping region.

20. The abdominal binder of claim 19, wherein the first compression band has an inner surface and an outer surface, the second compression band has an inner surface and an outer surface, one of the inner surface of the first compression band and the outer surface of the second compression band is provided with the loop surface of a hook-and-loop fastener, and the other is provided with the hook surface, so that the overlapping region adheres together when worn.
